(19) United States
(12) Patent Application Publication
(10) Pub. No.: US 2010/0125174 A1
(43) Pub. Date: May 20, 2010

(54) Remote Support of Implantable Medical Devices

(75) Inventors: Gregory C. Bevan, Canyon Country, CA (US); Elizabeth Bacon, Portland, OR (US); Eliot L. Ostrow, Sunnyvale, CA (US); George Walls, Valencia, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/273,492

(22) Filed: Nov. 18, 2008

(51) Int. Cl. A61B 5/00; A61N 1/08

(52) U.S. Cl. 600/300; 607/32; 607/60

(57) ABSTRACT

Exemplary medical devices and systems for providing remote support relating to implantable medical devices (IMDS) are described. One method generates a graphical user-interface (GUI) relating to an IMD on a local medical device configured to interrogate the IMD. The method also recreates the GUI on a remote medical device effective that a cursor of the GUI can be manipulated from both the local medical device and the remote medical device while selection of IMD parameter values is available only at the local medical device.

### FIELD OF THE INVENTION

The subject matter presented herein generally pertains to providing remote support relating to implantable medical devices (IMDs).

### BACKGROUND

Various implantable medical devices (IMDs) exist in the marketplace to treat a range of patient conditions. A variety of external medical devices are configured to communicate with the IMDs. For instance, a programmer can be utilized to retrieve data from an individual IMD. The data can be analyzed on the programmer and a clinician can change one or more parameter values in an attempt to enhance and potentially optimize a therapy supplied by the IMD. The described implementations provide support to the clinician in his/her programming decisions.

### SUMMARY

Exemplary medical devices and systems for providing remote support relating to implantable medical devices (IMDs) are described. One method generates a graphical user-interface (GUI) relating to an IMD on a local medical device configured to interrogate the IMD. The method also recreates the GUI on a remote medical device effective that a cursor of the GUI can be manipulated from both the local medical device and the remote medical device while selection of IMD parameter values is available only at the local medical device.

### BRIEF DESCRIPTION OF THE DRAWINGS

Features and advantages of the described implementations can be more readily understood by reference to the following description taken in conjunction with the accompanying drawings. In the description that follows, like numerals or reference designators will be used to reference like parts or elements wherever feasible.

FIG. 1 is a diagram of an exemplary system that includes a local device operated by a local clinician and a remote device operated by a remote clinician whereby information displayed locally is also displayed remotely.

FIG. 2 is a diagram of the system of FIG. 1 where the remote clinician can control a cursor of the local device.

FIG. 3 is a diagram of the system of FIGS. 1 and 2 illustrating a control operation.

FIG. 4 is a diagram of a device configured to communicate with an implantable medical device (IMD) and to program an IMD.

FIG. 5 is a diagram of a local device and associated graphical user interface (GUI) and a remote device and associated GUI where each GUI includes a text box for communication between operators of the local and remote devices.

FIG. 6 is a diagram of an exemplary system that allows a team of clinicians to participate in care of a patient with an IMD where the team includes at least one local clinician with exclusive control over programming of the IMD (see, e.g., the “program changes” control button 412 of FIG. 4).

FIG. 7 is a functional block diagram of an exemplary external medical device illustrating basic elements that are operable to provide remote support relating to implantable medical devices (IMDs) in accordance with some implementations.

FIG. 8 is a flowchart of an exemplary method for providing remote support relating to implantable medical devices (IMDs) in accordance with some implementations.

### DETAILED DESCRIPTION

### Overview

Various exemplary techniques, methods, devices, systems, etc., described herein pertain to remote support scenarios involving implantable medical devices (IMDs). For instance, a clinician (hereinafter local or first clinician) can utilize an external medical device to interrogate an (IMD). The interrogation by the external medical device can acquire information indicative of a patient's condition, IMD settings, etc., and then display the information for review by the local clinician. Upon display, the local clinician may have questions about the patient's status. For example, the local clinician may have questions about how to interpret patient information and/or how to adjust patient therapies implemented by the IMD and answers to such questions may require contacting another clinician, a device representative, or other person. As described herein, various exemplary technologies allow content from a local external medical device to be displayed on a remote device with optional controls for manipulation of the information and with restrictions that require certain actions to be taken by a local clinician only.

For example, an exemplary system allows a clinician (hereinafter remote or second clinician) at a remote site to view information and graphics viewed by the local clinician. Such an approach can assist the local clinician to take an appropriate course of action, such as adjusting a patient's therapy. As many local clinicians welcome support if they can maintain ultimate control over the patient, various implementations allow the remote clinician only selective control (i.e., restricted control) over the local external medical device's display. According to this scheme, the local clinician retains sole authority to implement any programming changes to the IMD. To facilitate communication, a system may include a dialog box at the external medical device and a dialog box at the remote device to facilitate textual correspondence between the local clinician and the remote clinician.

### Exemplary Systems

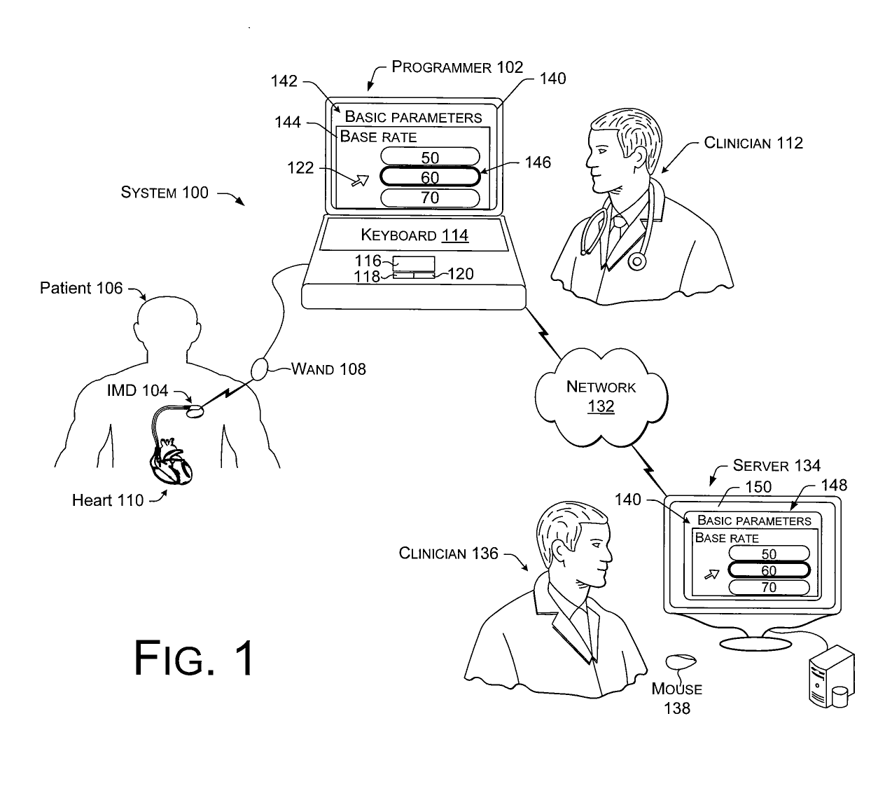

FIGS. 1-4 collectively illustrate a system 100 that enables remote support in implantable medical device (IMD) scenarios. System 100 includes an exemplary external medical device manifested as a programmer 102. An IMD 104 is positioned in a patient 106. The programmer 102 is configured to communicate with IMD 104 via a telemetry wand 108 or other mechanism. In this instance, IMD 104 functions as an implantable cardiac therapy device (ICTD) for a patient's heart 110. Although the IMD 104 is illustrated as an ICTD, the IMD 104 may be configured in a variety of ways, such as an implantable cardiac monitor that monitors heart activity, an implantable neurological device that monitors and/or stimulates nerves, and other implantable devices used for diagnostic and/or therapeutic purposes.

During a programming session, a first clinician 112 of programmer 102 can interrogate the IMD 104 to obtain IMD-related data. A programming session can be thought of as anytime IMD-related or patient data is exchanged between the IMD and an external medical device. In some cases the IMD-related data can include data sensed by the IMD 104 and/or any other data related to the IMD and/or the patient. The programmer 102 can offer processing and analysis of the IMD-related data and/or can display various aspects of the IMD-related data. The first clinician 112 can control the programmer via various input devices such as keyboard 114, touch pad or glide pad 116, and left and right click buttons 118, 120 to have specific IMD-related data displayed. In this case, control of programmer 102 is achieved by controlling a cursor 122 via the keyboard, touch pad and/or the left and right click buttons. In other implementations a mouse or other mechanism can be utilized to control the cursor 122. Still other implementations can employ a touch sensitive screen or display effective that the first clinician can engage the touch sensitive screen to control the programmer 102.

Network 132 allows data transfer between programmer 102 and a second external medical device in the form of a server 134. A second clinician 136 can view the data on server 134. As will be described in more detail below, the second clinician can selectively manipulate the data via an input device. In this case, the second clinician's input device is in the form factor of mouse 138. Implementations can alternatively or additionally include other input devices for the second clinician. As used herein descriptive terms are employed relative to the patient. For instance, programmer 102 is local to the patient and server 134 is remote relative to the patient.

FIG. 1 offers an example of a graphical user-interface (GUI) that can be generated from IMD-related data. In this case the GUI can be generated on a display area 140 of programmer 102 during a programming session of the patient's IMD. The GUI is manifested as a basic parameters screen 142 that includes a base rate parameter window 144. Three base rate parameter values “50”, “60”, and “70” are listed in base rate parameter window 144 in individual selectable dialog boxes. Further, the present base rate parameter value is “60” as indicated by shading designated generally at 146.

The display area 140 of programmer 102 is also shown on server 134 in a support window 148. In this case, support window 148 occupies a subset of the server's display area so that a portion 150 of the server's display area remains visible for the server's content. In other configurations, the support window can occupy all of the server's display area.

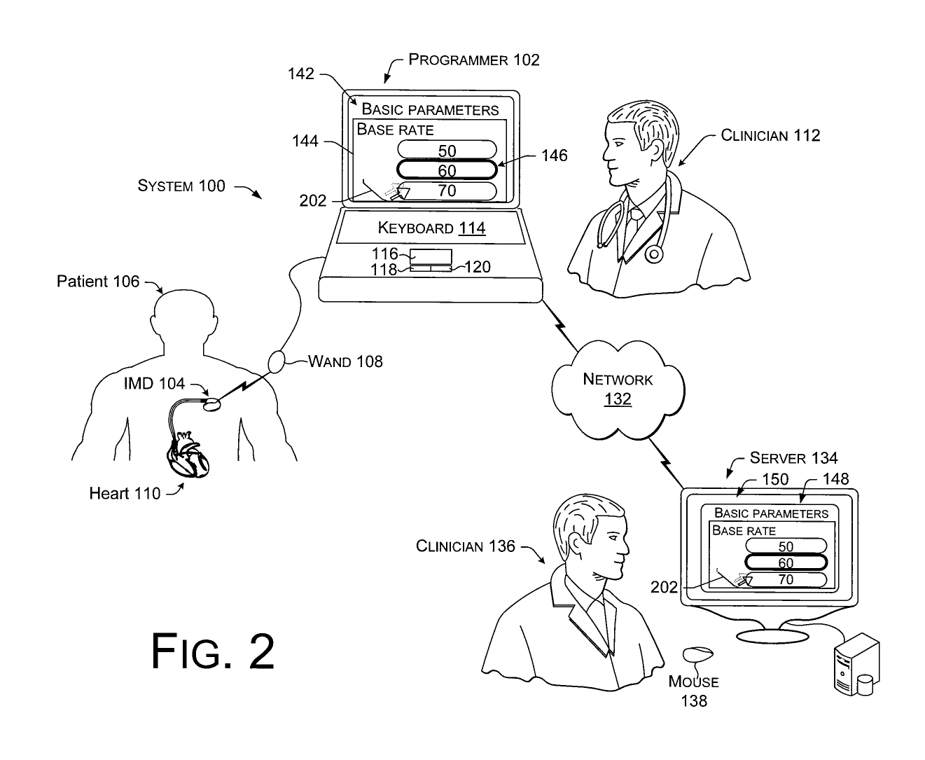

Assume for purposes of explanation that patient 106 is complaining of feeling poorly when exercising and that clinician 112 has brought up the basic parameter screen 142 responsive to the patient's complaints. Further, the first clinician 112 is considering changing the base rate parameter value to address the patient's complaints but is unsure of an appropriate strategy. The first clinician can communicate with the second clinician about the patient's clinical scenario. For instance, the clinicians can communicate orally or in writing over network 132 or via an independent mechanism. Assume for purposes of example that second clinician 136 recognizes that the base rate parameter value should be changed from “60” to “70” to address the patient's complaints. This scenario is continued below in relation to FIG. 2.

FIG. 2 illustrates movement of cursor 122 on basic parameter screen 140 on both the programmer 102 and the server 134. The cursor movement is indicated generally at 202 on programmer 102 and on server 124. The cursor movement culminated with the cursor being positioned over the “70” dialog box of the base rate window 144. The cursor movement can be accomplished from the programmer 102 and/or from the server 124. Assume that in this instance, the cursor movement was made by the second clinician on server 134 to aid the first clinician in addressing the patient's symptoms.

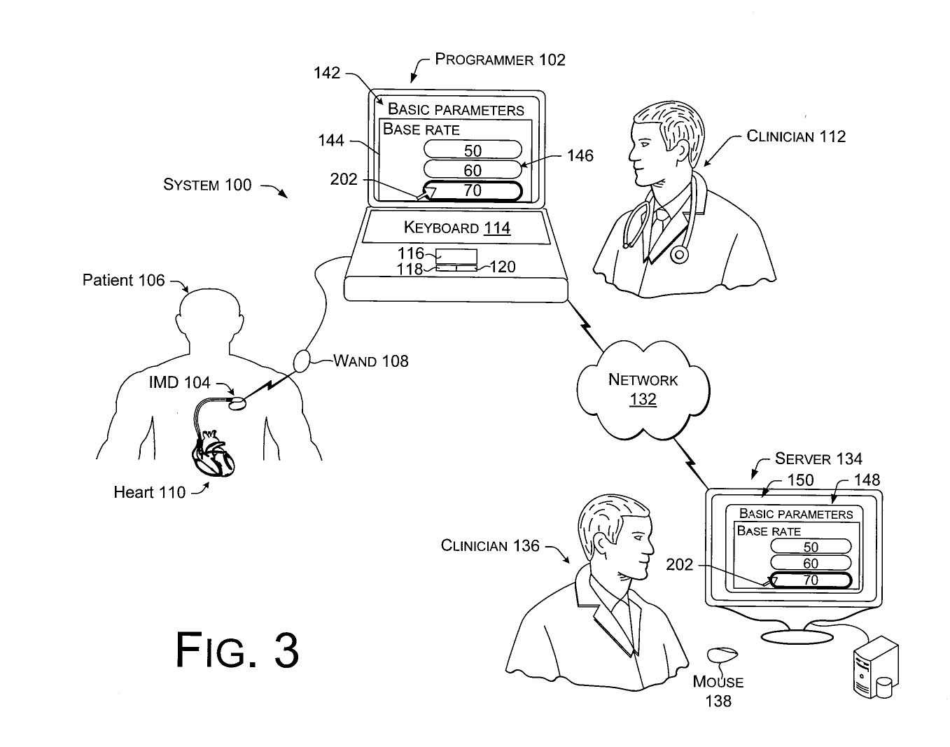

FIG. 3 illustrates selection of the value “70” dialog box within base rate parameter window 144 to alter the patient's treatment. The selection is evidenced on both the programmer 102 and the server 134. In this case the selection is indicated by shading of the dialog box associated with the parameter value of “70” and the removal of shading from the dialog box associated with the parameter value of “60”. In contrast to cursor movement which can be accomplished via either of the programmer 102 or the server 134, in this implementation, selection of an element such as a dialog box can be accomplished only on the programmer 102. Accordingly, while the second clinician can aid the first clinician in the decision making process ultimate authority and responsibility for any changes to the patient's therapy remains with the first clinician.

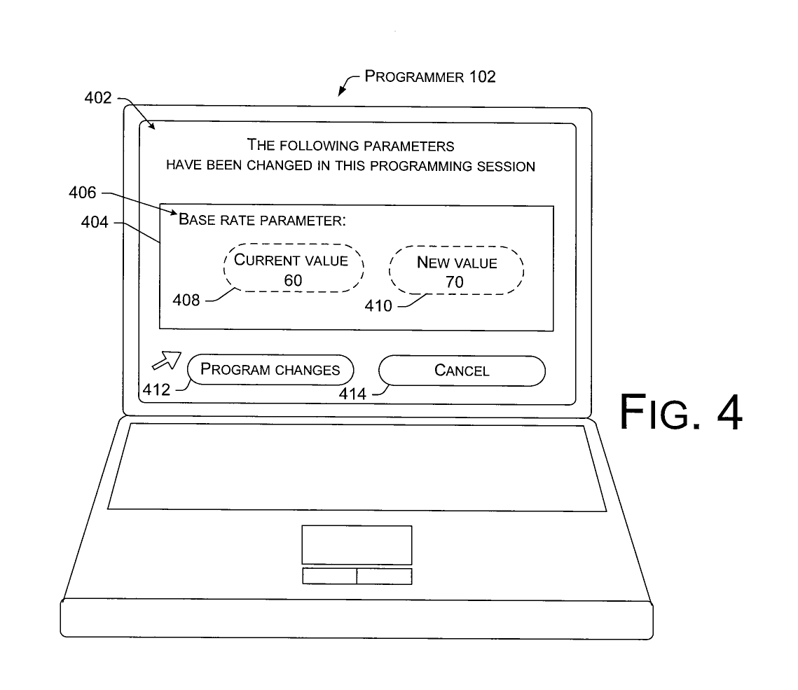

In another implementation, selection of elements such as parameter values and/or other programming changes can be made from either the programmer 102 or the server 134. For instance, selection of the “70” base rate value can be achieved on the programmer via the left or right click buttons 118, 120 or a key such as a “return” key of the keyboard 114. Similarly, the selection can be made via the server's mouse 138 or the server's keyboard (not shown). In this configuration final approval of the selections is reserved for the first clinician 112. An example relating to final approval of the selections is described in relation to FIG. 4.

FIG. 4 shows an example of a screen 402 on programmer 102 that allows the first clinician final approval for pending programming changes. Screen 402 lists the pending programming changes in a preview window 404. In this case the pending changes include the base rate parameter indicated at 406. A current value of “60” is listed for the base rate parameter at 408. A new or pending value of “70” is listed for the base rate parameter at 410. The first clinician can review the pending changes. The first clinician can either program the changes via a program changes dialog box 412 or cancel the changes via a cancel dialog box 414. Screen 402 also can be displayed on server 134, but selection of the program changes and cancel dialog boxes 412, 414 can only occur via programmer 102. This implementation can offer more extensive control to the second clinician than is offered in some other implementations while maintaining the final decision making responsibility for the first clinician. Accordingly the second clinician can provide additional assistance to the first clinician. For instance, the second clinician can select various display elements such as icons and/or dialog box(es) to reach a relevant screen rather than only being able to move the cursor and then relying on the first clinician to “click” at the appropriate times. Accordingly, in this case the second clinician utilizing the server can walk the first clinician through a series of steps to change the IMD's programming while the first clinician maintains final control to implement or cancel the parameter changes. In one such case where the programmer's screen 402 is a touch sensitive screen, both the first and second clinicians can be allowed to manipulate the cursor etc, but final selections can only be made by physically engaging the touch sensitive screen. In such a case only the first clinician is actually proximate the touch sensitive screen and can thereby make the final selections.

In a particular example, the local programmer 102 is the only device configured to display a control such as the “Program Changes” button 412. This approach ensures that the ultimate decision to program an IMD occurs locally and risk of error by a remote care provider is non-existent as any remote device is either prohibited or otherwise not configured to display such a programming control option. In another example, a remote device may display the button 412 but only as an inactive graphic. In this example, the local device includes the only active control, in the system, for making any changes to an IMD (e.g., setting a programmable parameter for delivery of a cardiac resynchronization therapy, disabling a feature, adjusting a shock energy, etc.).

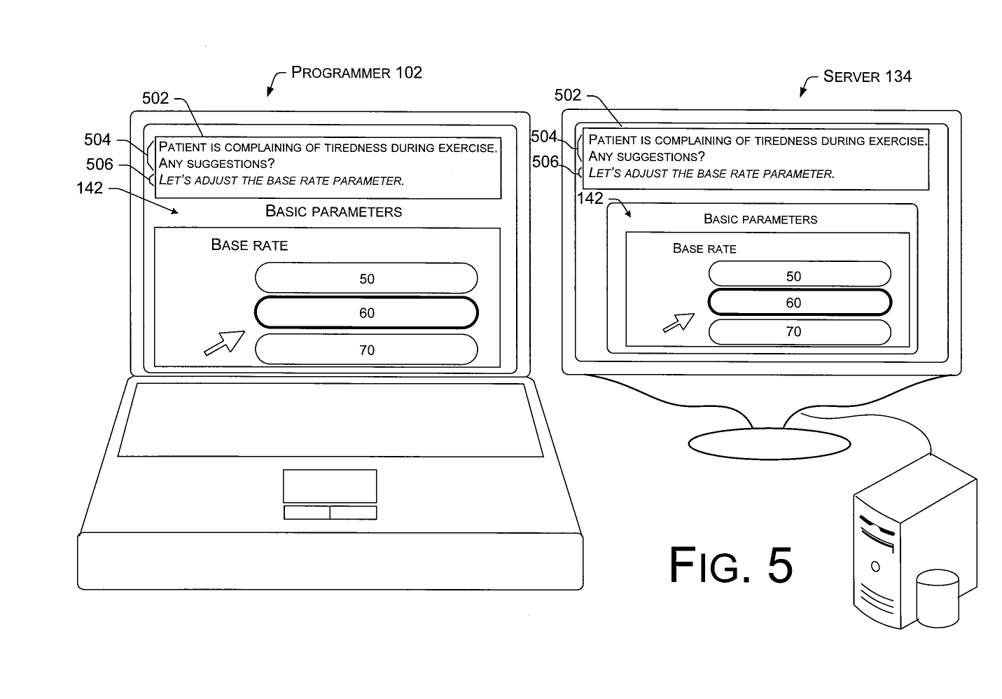

FIG. 5 shows another implementation relating to remote support. As described previously, content from the programmer 102, such as the basic parameters screen 142, can be displayed on both the programmer 102 and the server 134. Further, in this implementation a dialog box 502 is displayed on both the programmer 102 and the server 134. Dialog box 502 allows either of the first and second clinicians to type in text that can be seen by the other of the clinicians. Allowing text correspondence to be exchanged between the local and remote medical devices (in this case the programmer and the server) regarding the patient's IMD can enhance patient treatment. For instance, if the first clinician that is operating the programmer encounters a patient scenario that he/she is unsure how to treat, then the first clinician may want to be able to discretely obtain advice from the second clinician. For example, in some cases the first clinician may be uneasy about making a telephone call to the second clinician in front of the patient.

Dialog box 502 allows the clinicians to engage in a textual conversation while viewing the patient information on the respective programmer 102 and server 134. Such an implementation may increase a likelihood of the first clinician utilizing the expertise of the second clinician thereby enhancing patient treatment and decreasing mistakes and/or underutilization of the features of the IMD that the first clinician may not fully understand. For instance, in the illustrated example, as indicated generally at 504 the first clinician has described the patient's condition and requested suggestions from the second clinician. The second clinician responds “let's adjust the base rate parameter” as indicated at 506. In such a scenario, the dialog box can allow the second clinician to inform the first clinician what he/she intends to do prior to taking any action relating to the programmer's display such as switching screens and/or selecting parameter values. Accordingly, the dialog box can facilitate a meeting of the minds of the first and second clinicians about what they are trying to accomplish via the programmer's display.

In this case, text dialog box 502 is displayed on areas of programmer 102 and server 134 that are not presently utilized in displaying other IMD related content. In other configurations, the text dialog box can be superimposed over other GUI content related to the IMD. In some instances the text dialog box can be established in a fixed location on the display. In other configurations, the text dialog box is configured to be moved as desired such as by dragging-and-dropping.

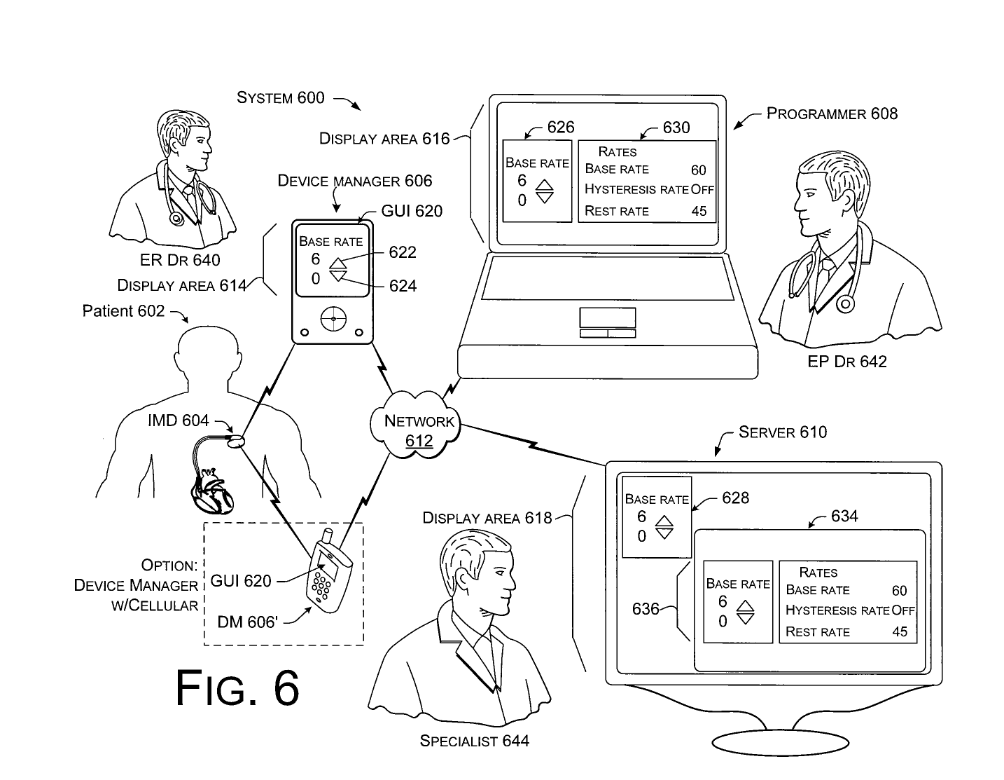

FIG. 6 shows a system 600 for providing remote support to a patient who has an IMD. In this case patient 602 has an IMD 604. System 600 includes external medical devices that can communicate with the IMD and/or process IMD-related data. In the present example, the system 600 includes three external medical devices (e.g., a device manager 606 or a cell-phone device 606′, the programmer 608 and the server 610). The device manager 606 or the cell-phone device 606′ is in close enough proximity to patient 602 to interrogate the patient's IMD 604. The device manager 606 or the cell-phone device 606′ can communicate with a remote medical device in the form of programmer 608 and/or a centralized medical device in the form of server 610 via a network 612. The device manager, programmer and server include display areas 614, 616, and 618 respectively for displaying IMD-related data.

Device manager 606 (or cell-phone device 606′) can be utilized to communicate IMD-related data to and from the IMD 604 sufficient to allow the device manager to reprogram the IMD. In this case, device manager 606 (or cell-phone device 606′) is configured to display IMD-related data on a GUI 620 that occupies display area 614. In this case, GUI 620 occupies all of display area 614. In other instances, the GUI can occupy a lesser subset of the display area. For purposes of example, the illustrated GUI 620 relates to a base rate parameter. The current base rate parameter value is listed as “60”. The GUI also includes a user-selectable up button 622 for increasing the base rate parameter value and a user-selectable down button 624 for decreasing the base rate parameter value.

The device manager's GUI 620 and/or display area 614 can also be displayed on both programmer 608 and server 610 as indicated generally at 626 and 628, respectively. In this case, the GUI occupies all of the display area so the two terms can be used interchangeably. Further, programmer 608 can be configured to display other IMD-related data in addition to the device manager's GUI 620. In this case, the additional IMD-related data is represented as rates window 630. For the cell-phone device 606′, the GUI 620 may be appropriately sized and displayed with various options for scrolling or paging (e.g., CSS or other form of display techniques).

Server 610 can be configured to display the display areas of both the device manager 606 (or cell-phone device 606′) and the programmer 608 on its display area 618. In this case, the device manager's display area 614 is displayed on the server as indicated at 628 and the programmer's display area 616 is displayed on the server as indicated generally at 634. In other configurations, the server can display only GUI's occupying the display area 614 of the device manager and/or the display area 616 of programmer rather than the entire display area. For instance, the GUI indicated generally at 636 can be included on server 610 rather than the programmer's entire display area that is indicated at 634. Stated another way, the server can be configured to display content and/or display area of both the device manager 606 (or cell-phone device 606′) and the programmer 608.

In some cases the device manager 606 (or cell-phone device 606′) can be utilized to provide a first or basic level of support for the patient's IMD, while the programmer 608 and the server 610 provide enhanced secondary and tertiary levels of support. For example, consider a usage scenario where the device manager is employed by an emergency room (ER) doctor 640 at a rural hospital. In this usage scenario the programmer is employed by the patient's electrophysiologist (EP) 642 and the server is employed by a specialist 644 in IMD function at a facility operated by the manufacturer of the IMD. In such a scenario, the device manager 606 offers a basic functionality that allows the ER doctor to view and/or adjust at least some patient-related data. For instance, the device manager can allow the ER doctor to adjust various parameter values of the IMD, such as the illustrated base rate parameter value. In some scenarios, the device manager provides a relatively robust functionality relative to the IMD, while in other scenarios the device manager offers a limited functionality,

In this case, the programmer 608 can offer a more robust functionality for processing and/or displaying IMD-related data than the device manager 606 (or cell-phone device 606′). In the illustrated configuration the programmer shows both the display of the device manager 606 (or cell-phone device 606′) as well as the rate information indicated at 630. Accordingly, the EP 642 can view patient related data that may not be accessible on the device manager. Finally, the server 610 allows the specialist 644 to view the content displayed on the device manager 606 (or cell-phone device 606′) as well as the content displayed on the programmer 608.

In some implementations, either or both of the EP 642 and the specialist 644 can control device manager 606 (or cell-phone device 606′) to aid the ER doctor 640 in making appropriate programming changes to the IMD 604. For example, the device manager 606 can be controlled via either the programmer 608 or the server 610. In some instances, the device manager can be selectively controlled via the server and/or the programmer. For instance, the device manager 606 (or cell-phone device 606′) can be selectively controlled via interaction with the device manager's GUI that is displayed on programmer 608 and the server 610. Manipulations received at the server or programmer are relayed to the device manager via network 612. Ultimate authority to implement IMD programming changes can be reserved for one of the ER doctor, the EP, or the specialist. For instance, the patient's EP may be most familiar with the patient's condition. In such a scenario, approval of any programming changes to the IMD can only be received at the programmer 608 so that the EP 642 retains ultimate authority and responsibility for the patient. In another case, ultimate authority for programming changes to the IMD can be reserved for the device manager 606 (or cell-phone device 606′) since it is proximate the patient. In this latter configuration, the ER doctor 640 is treating the patient and retains ultimate authority and responsibility for the patient.

### Exemplary External Medical Device Configuration

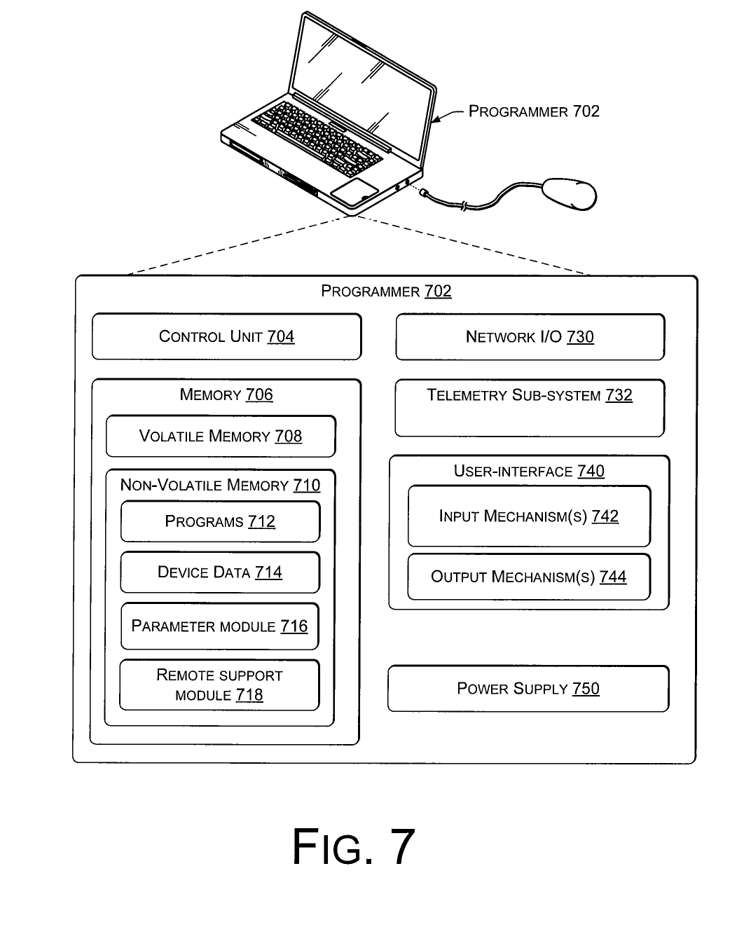

FIG. 7 describes functional components of an exemplary external medical device in the form of a programmer 702 in more detail. The described components can also be implemented in other external medical device configurations such as programmer 102, server 134, and/or device manager 606 described in relation to FIGS. 1-6, among others. The skilled artisan should recognize other devices that are compatible with the concepts described above and below. In this instance, programmer 702 includes a processing or control unit 704 and memory 706. The control unit 704 controls operations carried out by the programmer 702, such as programming an IMD, gathering data from the IMD, and/or carrying out various testing or diagnostic functions. Memory 706 includes both volatile memory 708 (e.g., RAM) and non-volatile memory 710 (e.g., ROM, EEPROM, Flash, disk, optical discs, persistent storage, etc.).

Programs, operating parameters, and algorithms 712, which are used in controlling the programming and testing functions, may be stored in memory 706. When a program is running, various instructions are loaded into volatile memory 708 and executed by control unit 704. IMD-related data or device data 714 collected from the IMD may be stored in memory 706 for subsequent analysis and/or transfer to other medical systems.

In this particular configuration, a parameter module 716 and a remote support module 718 are also stored in memory 706. Parameter module 716 is configured to determine a current parameter setting of the IMD as well as alternative values to which the parameter can be adjusted.

Remote support module 718 is configured to exchange data between programmer 702 and other medical devices to enable remote support. Examples of other medical devices include servers 134, 610 described in relation to FIGS. 1-3 and 5-6 and device manager 606 described above in relation to FIG. 6. In some configurations, the remote support module exchanges actual IMD-related data that is displayed on the programmer to other medical devices to allow the IMD-related data to be recreated on the other medical devices. In other configurations, the remote support module can send image data sufficient to recreate a GUI displayed on the programmer without sending the underlying IMD-related data conveyed by the GUI. In other words image data is sent sufficient to recreate the “look” of the GUI and not actually the GUI itself. Still other configurations can send some combination of image data and underlying IMD-related data.

The remote support module 718 can further be configured to receive input from a remote medical device such as a server and to cause the display of the programmer to be updated to reflect the received input. In some instances the remote support module can offer a degree of selectivity relating to the input received from another medical device. For instance, the remote support module can distinguish received input relating to cursor movement from received input relating to parameter value selection. In such a case, the remote support module can implement the cursor movement, but disallow the parameter value selection. The skilled artisan should recognize other variations. Further, while the current implementation involves remote support modules operating on individual medical devices, other implementations may be web-based where data processing tends to occur at a centralized location. Medical devices having various degrees of robustness can function as input/output devices for the processed data communicated from the central location.

The programmer 702 may further be equipped with a network I/O connection 730 to facilitate communication with a network and/or other medical devices such as a server(s). The network I/O 722 may be a wire-based connection (e.g., network card, modem, etc.) or a wireless connection, such as a Bluetooth device.

The programmer 702 can be equipped with a telemetry sub-system 732 that manages communications between programmer 702 and an IMD. The telemetry sub-system 732 can include telemetry hardware such as telemetry wands and/or other telemetry mechanisms for communicating with the IMD.

The programmer 702 may also include a user interface 740 which includes one or more user input mechanism(s) 742 and one or more output mechanisms 744. Input mechanisms allow user input to be received by the programmer. Examples of mechanisms for user input include, but are not limited to keypads, buttons, selection wheels, touch pads, touch screens or touch-sensitive screens, and voice recognition systems, among others. Output mechanisms 744 allow information to be provided from the programmer for user observation. The output mechanisms generate signals such as audio and/or visual signals that convey IMD-related data. Examples of output mechanisms include, but are not limited to, monitors, LEDs, speakers, and/or printing mechanisms, among others. For purposes of characterization, distinct input and output mechanisms are described, but in some instances, a single mechanism performs both functions. For instance, the user interface can be manifested as a touch-sensitive screen which performs both input and output functions.

The components illustrated in FIG. 7 are interconnected via one or more buses (not shown) and are powered by a power supply 750. Further, while aspects of programmer 702 are described in relation to modules implemented by programmer 702, various modules could alternatively or additionally be implemented as freestanding components such as application specific integrated circuits (ASIC). Additionally, various aspects of the methods and systems described throughout this disclosure may be implemented in computer software or firmware as computer-executable instructions. The instructions can be stored on any computer-readable storage media. When executed, these instructions direct the programmer to perform various functions and tasks described above and below.

### Operation

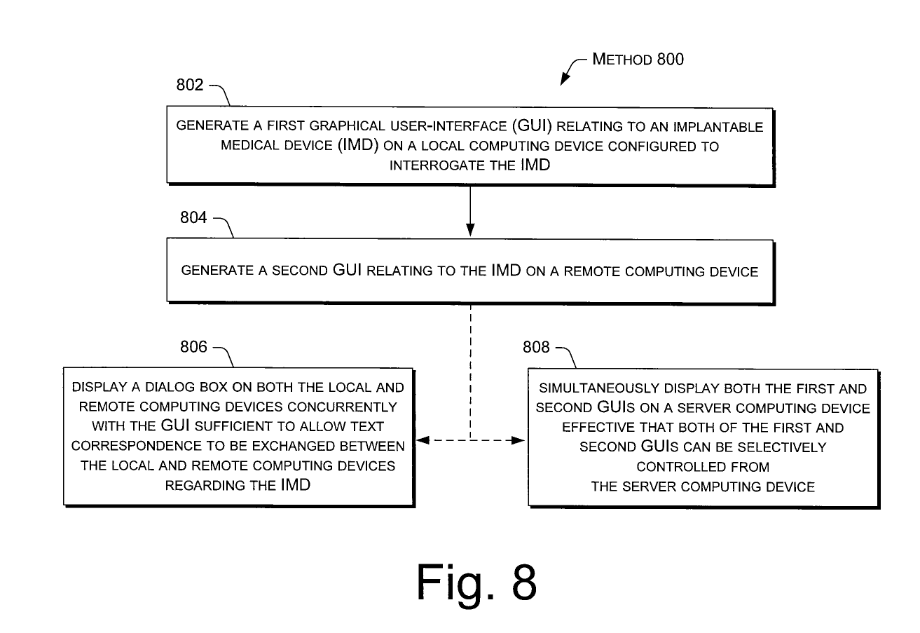

FIG. 8 shows an exemplary method or technique 800 for providing remote support relative to programming an implantable medical device (IMD). This method 800 may be implemented in connection with any suitably configured external medical devices and/or systems of external medical devices and/or IMDs. Non-limiting examples of devices and/or systems upon which the method can be implemented are described above in relation to FIGS. 1-7.

The order in which the method 800 is described is not intended to be construed as a limitation, and any number of the described blocks can be combined in any order to implement the method, or an alternate method. Furthermore, the methods can be implemented in any suitable hardware, software, firmware, or combination thereof such that a computing device can implement the method. In one case, the method is stored on a computer-readable storage media as a set of instructions such that execution by a computing device, such as an external medical device, causes the computing device to perform the method.

At block 802, a first graphical user-interface (GUI) that relates to an implantable medical device (IMD) is generated on a local medical device configured to interrogate the IMD. The local medical device can be any medical device configured to communicate with the IMD. For instance, the local medical device can be in the form factor of a device manager, personal computer, programmer, or the like. The GUI can show various parameters that relate to the IMD. In some instances, a first clinician or user of the local medical device can change values of one or more parameters. The local medical device can then communicate the changed parameter values to the IMD. Stated another way, the IMD can be reprogrammed to reflect the changed parameter values.

At block 804, a second GUI relating to the IMD is generated on a remote medical device. In some scenarios the remote medical device is configured to be operated in a supporting role to the local medical device. For instance, a second clinician operating the remote medical device may be more highly trained and/or potentially more knowledgeable about IMD functioning and/or the patient's condition than the first clinician. To this end, the remote medical device can be configured to allow the second clinician to offer remote support to the first clinician regarding the patient's IMD. In some cases, the GUI of the local medical device is recreated on the remote medical device effective that at least some GUI features or elements are controllable from either or both of the local medical device and the remote medical device. For instance, in some configurations a cursor of the GUI can be manipulated from both the local medical device and the remote medical device. In another example, a window relating to a particular parameter can be opened from either the local or remote medical devices.

Control of other GUI features can be restricted to the local medical device to maintain ultimate decision making authority with the first clinician. For example, in some configurations while cursor manipulation is shared, selection of IMD parameter values is available only at the local medical device. Still other configurations allow cursor movement and selection of GUI elements from both the local and remote medical devices. However, any resulting parameter changes to the IMD's programming can only be selected via the local medical device. For instance, the parameter changes can be listed on a summary screen which offers the option of programming the parameter changes or cancelling the changes. Selection at the summary screen can only be made via the local medical device.

Blocks 806 and 808 offer two variations on the concepts described above. At block 806 a dialog box can be displayed on both the local and remote medical devices concurrently with the GUI. The dialog box can allow text correspondence to be exchanged between the local and remote medical devices regarding the IMD. The dialog box facilitates communication between the first and second clinicians to promote appropriate patient therapy. Further, the dialog box offers discreet communication between the first and second clinicians in the presence of the patient. The first clinician is more likely to be candid and/or to request help from the remote clinician when using the dialog box compared to a telephone conversation between the two clinicians in the presence of the patient.

At block 808 the method simultaneously displays both the first and second GUIs on a server medical device effective that both of the first and second GUIs can be selectively controlled from the server medical device. As used herein the term “server” simply means a medical device associated with or operated by a user skilled in IMD function and/or patient therapy. In some configurations the server medical device displays all of the displayed content of the local and remote medical devices. In other implementations only the GUIs from the local and remote medical devices are displayed on the server. Displaying the content of the local and remote medical devices on the server allows the server's user to oversee the actions of the first and second clinicians and/or allows the server's user to aid in reprogramming the patient's IMD. For instance, in some scenarios, the server's user has selective or unlimited control of both the local and remote medical devices via the server medical device.

### CONCLUSION

Exemplary techniques, methods, devices, systems, etc., for providing remote support relative to programming an implantable medical device (IMD) are described above. Although techniques, methods, devices, systems, etc., have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods, devices, systems, etc.

What is claimed is:

1. A method comprising:
generating a graphical user-interface (GUI) relating to an implantable medical device (IMD) on a local medical device configured to interrogate the IMD;
recreating the GUI on a remote medical device effective that a cursor of the GUI can be manipulated from both the local medical device and the remote medical device while selection of IMD parameter values is available only at the local medical device;
selecting a IMD parameter value using the local medical device, the selected parameter value indicated by manipulation of the cursor by the remote medical device; and
communicating the selected parameter value from the local medical device to the IMD.

2. The method of claim 1, wherein either of the local or remote medical devices can be utilized to move the cursor over dialog boxes associated with various IMD parameters and wherein only the local medical device allows for selection of an individual dialog box.

3. The method device of claim 1, wherein the recreating comprises recreating an entire display area of the local medical device.

4. The method device of claim 1, further including displaying a text box on both of the local and remote medical devices to allow textual communication between a user of the local medical device and a user of the remote medical device.

5. A method comprising:
generating a graphical user-interface (GUI) relating to an implantable medical device (IMD) on a local medical device configured to interrogate the IMD; and,
recreating the GUI on a remote medical device effective that GUI elements can be selected from both the local and remote medical devices while any resulting parameter changes to the IMD's programming can only be selected via an active control of the local medical device.

6. The method of claim 5, wherein the parameter changes are reflected on a session parameter changes summary window and wherein the parameter changes can only be programmed via a selection on the session parameter changes summary window received from the local medical device.

7. The method of claim 6, wherein the recreating provides equivalent functionality on both the local and remote medical devices except for the session parameter changes summary window.

8. One or more computer-readable storage media comprising computer-executable instructions that, when executed perform acts comprising:
generating a graphical user-interface (GUI) relating to an implantable medical device (IMD) on a local medical device configured to interrogate the IMD;
recreating the GUI on a remote medical device effective that GUI elements can be selected from both the local and remote medical devices;
displaying a dialog box on both the local and remote medical devices concurrently with the GUI sufficient to allow text correspondence to be exchanged between the local and remote medical devices regarding the IMD.

9. The computer-readable storage media of claim 8, wherein the displaying comprises displaying the dialog box on a display area of the local medical device and a display area of the remote medical device effective that dialog box does not obstruct displayed content relating to the IMD.

10. The computer-readable storage media of claim 8, wherein the displaying comprises superimposing the dialog box over at least a portion of the GUI.
